(19) United States
(12) Patent Application Publication
(10) Pub. No.: US 2016/0107141 A1
(43) Pub. Date: Apr. 21, 2016

(54) SOLID SUPPORT INCLUDING A POLYMER AND USE THEREOF

(71) Applicant: Samsung Electronics Co., Ltd., Suwon-si (KR)

(72) Inventors: Myoungsoon Kim, Anyang-si (KR); Gahee Kim, Yongin-si (KR); Youngwan Ha, Seoul (KR)

(21) Appl. No.: 14/884,548

(22) Filed: Oct. 15, 2015

(30) Foreign Application Priority Data
Oct. 15, 2014 (KR) 10-2014-0139066

(51) Int. Cl. B01J 20/286; G01N 33/545; B01J 20/26

(52) U.S. Cl. CPC B01J 20/286; B01J 20/265; G01N 33/545

(57) ABSTRACT

A solid support with a polymer, and a method of using the solid support are provided.

### RELATED APPLICATIONS

This application claims the benefit of Korean Patent Application No. 10-2014-0139066, filed on Oct. 15, 2014, in the Korean Intellectual Property Office, the entire disclosure of which is hereby incorporated by reference.

### BACKGROUND

1. Field

The present disclosure relates to polymers containing a group having a fluorocarbon and methods of using the same.

2. Description of the Related Art

There are known methods of binding biomolecules to a support or separating biomolecules therefrom. For example, protein separation devices including a ligand protein immobilized on a support are known. However, when such methods, which are used to separate protein that is specifically bound to a ligand, are used, non-specific binding of biomolecules to a support needs to be prevented to enhance binding and detection efficiencies.

Traditionally, to decrease a non-specific binding between a support and protein, a method of blocking a site of a support at which a non-specific binding occurs by using a blocking agent such as bovine serum albumin (BSA) is known.

Acrylate polymers belong to a group of polymers. Acrylate monomers that may be used in acrylate polymers include acrylic acids, methyl methacrylates, and acrylonitriles. Examples of the acrylate polymers include polyacrylate, polymethacrylate, and polyacrylonitrile. In addition, acrylate polymers may be acrylic elastomers, acrylic fibers, acrylic paints, or acrylic resins.

### SUMMARY

Provided is solid support on which at least one polymer is immobilized, the at least one polymer comprising at least one of repeating units represented by Formula M1 and at least one of repeating units represented by Formula M2:

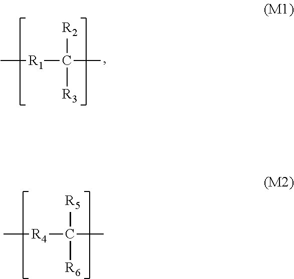

wherein, in Formulae M1 and M2,
R₁ and R₄ are each independently a bond or a substituted or unsubstituted C₁₋₂₀ alkyl, C₂₋₂₀ alkenyl, or C₂₋₂₀ alkynyl group;

R₂ and R₅ are each independently a hydrogen, a halogen, or a substituted or unsubstituted C₁₋₂₀ alkyl, C₂₋₂₀ alkenyl, or C₂₋₂₀-alkynyl group;

R₃ is W¹—X;
R₆ is W²—Y;
W¹ and W² are each independently a bond, —C(═O)—, —C(═S)—, —C(═O)O—, —C(═O)O—C(═O)—, —C(═O)NR⁷—, —C(═S)NR⁷—, —S(═O)—, or —S(═O)₂—,

R⁷ is H or a C₁₋₂₀ alkyl group;

X and Y are each independently selected from the group consisting of H, a fluorocarbon, and a material that specifically binds to one or more biomolecules.

Also provided is a method of binding biomolecules to the solid support, and a method of using the support to detect biomolecules in a sample.

Additional aspects will be set forth in part in the description which follows and, in part, will be apparent from the description, or may be learned by practice of the presented embodiments.

### BRIEF DESCRIPTION OF THE DRAWINGS

These and/or other aspects will become apparent and more readily appreciated from the following description of the embodiments, taken in conjunction with the accompanying drawings in which:

FIG. 1 is a graph of relative contact angles of poly(acrylic acid) (PAA), poly(methacrylate) (PMA), poly(methylsilane) (PMS), and polyethylmethacrylate (PEM);

FIG. 2 is a graph illustrating the results of bicinchoninic acid (BCA) assay on PAA, PMA, and PEM;

FIG. 3 is a graph of relative contact angles of CF₃(CF₂)₇CH₂NH₂ (FC1) and CF₃CF₂CF₂CH₂NH₂ (FC2);

FIG. 4 is an image illustrating the results of sodium dodecyl sulfate polyacrylamide gel electrophoresis (SDS-PAGE) on polymer-including solid supports;

FIG. 5 is a graph of band intensity resulting from SDS-PAGE on the polymer-including solid supports as magnetic beads after incubation with bovine serum albumin.

FIG. 6 is an image illustrating the results of SDS-PAGE on APpGBFC2, APpGB, and APpGFC2 after incubation with BSA-including buffer and streptavidin;

FIG. 7 is a graph of relative band intensity resulting from SDS-PAGE on the polymer-including solid supports after incubation with BSA; and

FIG. 8 is a graph of relative band intensity of streptavidin-solid support composites, resulting from SDS-PAGE on polymer-including solid supports after the incubation with streptavidin.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

According to an embodiment of the present disclosure, there is provided a solid support on which at least one polymer is immobilized, the at least one polymer including at least one repeating unit represented by Formula M1 and at least one repeating unit represented by Formula M2:

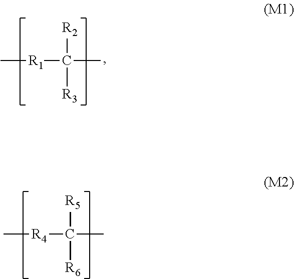

wherein, in Formulae M1 and M2,

R₁ and R₄ are each independently a bond or a substituted or unsubstituted C₁₋₂₀ alkyl, C₂₋₂₀ alkenyl, or C₂₋₂₀ alkynyl group;

R₂ and R₅ are each independently a hydrogen, a halogen, or a substituted or unsubstituted C₁₋₂₀ alkyl, C₂₋₂₀ alkenyl, or C₂₋₂₀ alkynyl group;

R₃ is W¹—X;

R₆ is W²—Y;

W¹ and W² are each independently deleted, —C(═O)—, —C(═S)—, —C(═O)O—, —C(═O)O—C(═O)—, —C(═O)NR⁷—, —C(═S)NR⁷—, —S(═O)—, or —S(═O)₂—;

R⁷ is H or a C₁₋₂₀ alkyl group; and

X and Y are each independently selected from the group consisting of H, a fluorocarbon, and a material that specifically binds to a one or more biomolecules.

In some embodiments of the polymer, R₁ and R₄ may be each independently a bond, for example, a simple single bond, a substituted or unsubstituted C₁₋₂₀ alkyl (for example, methyl, ethyl, propyl, isopropyl, butyl, sec-butyl, tert-butyl, pentyl, hexyl, heptyl, octyl, nonyl, decyl, or the like), a substituted or unsubstituted C₂₋₂₀ alkenyl, or a substituted or unsubstituted C₂₋₂₀ alkynyl; and

R₂ and R₅ may be each independently a hydrogen (H), a halo group, a substituted or unsubstituted C₁₋₂₀ alkyl (for example, methyl, ethyl, propyl, isopropyl, butyl, sec-butyl, tert-butyl, pentyl, hexyl, heptyl, octyl, nonyl, decyl, or the like), a substituted or unsubstituted C₂₋₂₀ alkenyl, or a substituted or unsubstituted C₂₋₂₀ alkynyl,

wherein a substituent for each of R₁, R₂, R₄, and R₅ may be a halogen atom, a C₁₋₂₀ alkyl group substituted with a halogen atom (for example, CCF₃, CHCF₂, CH₂F, CH₂Br, CH₂Cl, or CCl₃), a hydroxy group, a nitro group, a cyano group, an amino group, an amidino group, a hydrazine, a hydrazone, a carboxyl group or a salt thereof, a substituted sulfonic acid group or a salt thereof, a phosphoric acid group or a salt thereof, or a C₁₋₂₀ alkyl group, a C₁₋₂₀ alkoxy group, a C₂₋₂₀ alkenyl group, a C₂₋₂₀ alkynyl group, a C₁₋₂₀ heteroalkyl group, a C₆₋₂₀ aryl group, a C₆₋₂₀ arylalkyl group, a C₆₋₂₀ heteroaryl group, or a C₆₋₂₀ heteroarylalkyl group.

In some embodiments of the polymer, the number of repeating units represented by Formula M1 in a polymer immobilized on the solid support may be in a range of about 1 to about 300, for example, about 1 to about 250, about 1 to about 200, about 1 to about 180, about 10 to about 300, about 10 to about 250, about 10 to about 200, about 10 to about 180, about 30 to about 300, about 30 to about 250, about 30 to about 200, about 30 to about 180, about 50 to about 300, about 50 to about 250, about 50 to about 200, about 50 to about 180, about 70 to about 300, about 90 to about 250, about 100 to about 200, or about 100 to about 180, and the number of repeating units represented by Formula M2 of the polymer may be in a range of about 1 to 300, for example, about 1 to about 250, about 1 to about 200, about 1 to about 180, about 10 to about 300, about 10 to about 250, about 10 to about 200, about 10 to about 180, about 30 to about 300, about 30 to about 250, about 30 to about 200, about 30 to about 180, about 50 to about 300, about 50 to about 250, about 50 to about 200, about 50 to about 180, about 70 to about 300, about 90 to about 250, about 100 to about 200, or about 100 to about 180.

In some embodiments of the polymer, the fluorocarbon may be a fluoro-containing C₁₋₂₀ substituted or unsubstituted, linear or branched compound. For example, the fluorocarbon may be a fluoro-containing C₁₋₂₀ alkyl compound, a fluoro-containing C₁₋₂₀ carbonyl compound, a fluoro-containing C₁₋₂₀ alkoxy compound, or a combination thereof. The fluorocarbon may be a perfluorocarbon.

With regard to compounds, compositions, and methods referred to in conjunction with embodiments of the present disclosure, terminologies are defined as follows unless stated otherwise.

The term “alkyl” refers to a linear or branched monovalent saturated hydrocarbon group. Unless stated otherwise, the alkyl group may include about 1 to 10, about 1 to 8, about 1 to 6, about 1 to 4, or about 1 to 3 carbon atoms. Non-limiting examples of the alkyl group include methyl, ethyl, propyl (for example, n-propyl and isopropyl), butyl (for example, n-butyl, isobutyl, and t-butyl), pentyl (for example, n-pentyl, isopentyl, and neopentyl), n-hexyl, n-heptyl, n-octyl, n-nonyl, and n-decyl.

The term “alkenyl” refers to a linear or branched monovalent unsaturated hydrocarbon group with at least one carbon-carbon double bond. Unless stated otherwise, the alkenyl group may include about 2 to 10, about 2 to 8, about 2 to 6, about 2 to 4, or about 2 to 3 carbon atoms. Non-limiting examples of the alkenyl group include ethenyl, n-propenyl, isopropenyl, n-but-2-enyl, cyclohexenyl, and n-hex-3-enyl.

The term “alkynyl” refers to a linear or branched monovalent unsaturated hydrocarbon group with at least one carbon-carbon triple bond. Unless stated otherwise, the alkynyl group may include about 2 to 10, about 2 to 8, about 2 to 6, about 2 to 4, or about 2 to 3 carbon atoms. Non-limiting examples of the alkynyl group include ethynyl, n-propynyl, n-but-2-ynyl, and n-hex-3-ynyl.

The term “haloalkyl” refers to an alkyl group with at least one halogen substituent. Non-limiting examples of the haloalkyl group include —CF₃, —C₂F₅, —CHF₂, —CCl₃, —CHCl₂, and —C₂Cl₅. Unless stated otherwise, the haloalkyl group may include about 1 to 6, about 1 to 4, or about 1 to 3 carbon atoms.

The term “aryl” refers to a monocyclic or polycyclic aromatic hydrocarbon group. The polycyclic may include a fused ring (for example, naphthalene) and/or a unfused ring (for example, biphenyl). The polycyclic may include, for example, 2, 3, or 4 rings. Unless stated otherwise, the aryl group may include about 5 to 20, about 6 to 15, about 6 to 12, or about 6 to 10 carbocyclic atoms. Non-limiting examples of the aryl group include phenyl, naphthalenyl (for example, naphthalene-1-yl and naphthalene-2-yl), biphenyl, anthracenyl, and phenanthrenyl.

The term “cycloalkyl” refers to a non-aromatic carbocyclic group including a cyclic alkyl, alkenyl, or alkynyl group. The cycloalkyl group may be monocyclic or polycyclic. The polycyclic may include, for example, 2, 3, or 4 fused rings. Unless stated otherwise, the cycloalkyl group may include about 3 to 10, or about 3 to 7 cyclic carbon atoms. Non-limiting examples of the cycloalkyl group include cyclopropyl, cyclobutyl, cyclopentyl, cyclohexyl, cycloheptyl, cyclohexadienyl, cycloheptatrienyl, norbornyl, norcarnyl, and adamantyl.

The term “heterocycloalkyl” refers to a non-aromatic heterocyclic group including at least one ring-forming heteroatom selected from N, O, and S. The heterocycloalkyl group may have a monocyclic or polycyclic structure including, for example, 2, 3, or 4 fused rings. Non-limiting examples of the heterocycloalkyl group include morpholinyl, thiomorpholinyl, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, and thiazolidinyl. Unless stated otherwise, the heterocycloalkyl group may include about 3 to 10, about 3 to 7, about 5 to 7, or about 5 to 6 ring-forming atoms.

The term “heteroaryl” refers to a monovalent aromatic group including at least one heteroatom selected from N, O, and S as a ring-forming atom. The heteroaryl group may include a monocyclic or polycyclic structure. The polycyclic may include, for example, 2, 3, or 4 condensed rings. Unless stated otherwise, the heteroaryl group may include about 3 to 10, about 3 to 7, or about 3 to 5 cyclic atoms. The heteroaryl group may include 1, 2, or 3 heteroatoms. Non-limiting examples of the heteroaryl group include pyridyl, N-oxopyridyl, pyrimidinyl, pyrazinyl, pyridazinyl, triazinyl, furyl, quinolyl, isoquinolyl, thienyl, imidazolyl, furanyl, thiazolyl, indolyl, pyrryl, oxazolyl, benzofuryl, benzothienyl, benzothiazolyl, isoxazolyl, pyrazolyl, triazolyl, tetrazolyl, indazolyl, 1,2,4-thiadiazolyl, isothiazolyl, benzothienyl, purinyl, benzimidazolyl, and indolinyl.

The term “halo”, “halo group” or “halogen” refers to a fluoro, chloro, bromo, or iodo group.

The term “arylalkyl” refers to an alkyl group substituted with an aryl group. These aryl alkyl group are the same as defined above.

The term “heteroarylalkyl” refers to an alkyl group substituted with a heteroaryl group. These heteroaryl and alkyl groups are the same as defined above.

The term “substituted” used in the substituted alkyl group, the substituted alkoxy group, the substituted alkenyl group, the substituted alkynyl group, the substituted alkylene oxide group, the substituted cycloalkyl group, the substituted aryloxy group, and the substituted heteroaryl group may indicate that at least one hydrogen atom of the above mentioned groups is substituted with a halogen atom, a C₁₋₂₀ alkyl group substituted with a halogen atom (for example, CCF₃, CHCF₂, CH₂F, CH₂Br, CH₂Cl, or CCl₃), a hydroxy group, a nitro group, a cyano group, an amino group, an amidino group, a hydrazine, a hydrazone, a carboxyl acid group or a salt thereof, a sulfonic acid group or a salt thereof, a phosphoric acid group or salt thereof, or a C₁₋₂₀ alkyl group, a C₁₋₂₀ alkoxy group, a C₂₋₂₀ alkenyl group, a C₂₋₂₀ alkynyl group, a C₁₋₂₀ heteroalkyl group, a C₆₋₂₀ aryl group, a C₆₋₂₀ arylalkyl group, a C₆₋₂₀ heteroaryl group, or a C₆₋₂₀ heteroaryl alkyl group.

Examples of the C₁₋₂₀ alkyl group are methyl, ethyl, propyl, isobutyl, sec-butyl, tert-butyl, neo-butyl, iso-amyl, hexyl, or the like. At least one hydrogen atom in these alkyl groups may be substituted with those substituents described above in conjunction with the “substituted”.

Examples of the C₁₋₂₀ alkoxy group are methoxy, ethoxy, propoxy, or the like. At least one hydrogen atom in these alkoxy groups may be substituted with those substituents described herein in conjunction with the tem “substituted”.

Examples of the C₂₋₂₀ alkenyl group are vinylene, allylene, or the like. At least one hydrogen atom in these alkenyl groups may be substituted with those substituents as described herein in conjunction with the term “substituted”.

An example of the C₂₋₂₀ alkynyl group is acetylene. At least one hydrogen atom in the alkynyl group may be substituted with those substituents as described herein in conjunction with the term “substituted”.

Examples of the C₂₋₂₀ alkylene oxide group are ethylene oxide, propylene oxide, butylene oxide, or the like.

Examples of the C₃₋₂₀ cycloalkyl group are cyclopropyl, cyclobutyl, cyclopentyl, cyclohexyl, or the like. At least one hydrogen atom in these cycloalkyl groups may be substituted with those substituents as described herein in conjunction with the term “substituted”.

The C₆₋₂₀ aryl group may be used alone or in combination, and refers to an aromatic system including at least one ring. Examples of the C₆₋₂₀ aryl group are phenyl, naphthyl, or the like. At least one hydrogen atom in the aryl group may be substituted with those substituents as described herein in conjunction with the term “substituted”.

An example of the C₆₋₂₀ aryloxy group is a phenoxy group. At least one hydrogen atom in the aryloxy group may be substituted with those substituents as described herein in conjunction with the term “substituted”.

The C₆₋₂₀ heteroaryl group refers to a carbocyclic organic compound including at least one heteroatom selected from nitrogen (N), oxygen (O), phosphorous (P), and sulfur (S), the rest atoms of the ring all being carbon. An example of the C₆₋₂₀ heteroaryl group is pyridyl. At least one hydrogen atom in the heteroaryl group may be substituted with those substituents as described herein in conjunction with the term “substituted”.

The solid support may be of any shape. For example, the solid support may be in the form of a bead, a plate, or a well. For example, the solid support may be resin. The resin may be, for example, a magnetic particle. The solid support (or surface thereof) may be formed of a material that does not non-specifically bind to biomolecules or exhibits low binding to biomolecules. For example, the solid support may include a material selected from the group consisting of polyethylene, polypropylene, polybutylene, polyvinylchloride, polystyrene, acrylamide, agarose, cellulose, nitrocellulose, glass, polystyrene, polyethylene vinyl acetate, polypropylene, polymethacrylate, polyethylene, polyethylene oxide, polypolysilicate, polycarbonate, teflon, nylon, silicon rubber, polyanhydride, polyglycolic acid, polylactic acid, polyorthoester, polypropylfumerate, collagen, glycosaminoglycan, polyamino acid, and plastic.

The solid support may have a cross-sectional length of about 100 nm or greater. For example, the solid support may have at least one cross-sectional length of about 1000 nm, 10 μm, 100 μm, or 1000 μm or greater. In some embodiments, the solid support may have at least one cross-sectional length of about 100 nm to about 1000 μm, about 1000 nm to about 1000 μm, about 10 μm to about 1000 μm, about 100 μm to about 1000 μm, about 1 μm to about 1000 μm, about 1 μm to about 1000 μm, about 2 μm to about 1000 μm, about 100 nm to about 1000 μm, about 1000 nm to about 100 μm, about 1 μm to about 10 μm, about 1 μm to about 5 μm, or about 1 μm to about 7 μm.

In some embodiments, a plurality of polymers may be immobilized on the solid support. For example, about 2 or more, about 5 or more, about 10 or more, about 20 or more, about 50 or more, about 100 or more, about 200 or more, about 500 or more, about 1000 or more, about 2000 or more, about 5000 or more, or about 10,000 or more of the polymers may be immobilized on the solid polymer. In some other embodiments, about 2 to about 10,000, about 5 to about 10,000, about 10 to about 10,000, about 20 to about 10,000, about 50 to about 10,000, about 100 to about 10,000, about 200 to about 10,000, about 500 to about 10,000, about 1,000 to 10,000, about 2,000 to about 10,000, about 5,000 to about 10,000, about 2 to about 5,000, about 5 or about 5,000, about 10 to about 5,000, about 20 to about 5,000, about 50 to about 5,000, about 100 to about 5,000, about 200 to about 5,000, about 500 to about 5,000, about 1,000 to about 5,000, about 2,000 to about 5,000, about 2 to about 2,000, about 5 to about 2,000, about 10 to about 2,000, about 20 to about 2,000, about 50 to about 2,000, about 100 to about 2,000, about 200 to about 2,000, or about 500 to about 2,000, or about 1,000 to about 2,000 of the polymers may be immobilized on the solid support.

In some embodiments of the polymer, the biomolecule may be selected from a protein, a nucleic acid, and a sugar. For example, the biomolecule may be a protein. The term “protein” used herein refers to molecules that entirely or partially include polymers in which natural or non-natural amino acids are linked by amide bonds. The protein also includes a protein analog such as peptide nucleic acid (PNA). The term “analog” used herein is interpreted as a material including a side chain of a natural or non-natural amino acid on a molecular backbone thereof, like a natural protein including an amino acid side chain exposed to a molecular surface thereof. The protein may be a natural or non-natural protein.

In some embodiments of the polymer, the material that specifically binds to a biomolecule may be any material able to bind to a biomolecule, for example, it may be a capture molecule. The capture molecule may be selected from the group consisting of a protein, a nucleic acid, and a sugar. For example, the capture material may be an antibody, an antigen against an antibody, a receptor against a ligand, a ligand against a receptor, a substrate or inhibitor of an enzyme, or an enzyme against a substrate or inhibitor. For example, the capture material may include Protein G, Protein A, lectin, an antibody, avidin, streptavidin, a receptor protein, or a combination thereof.

The polymer may be synthesized to include the repeating units of Formulae M1 and M2 where W¹ and W² is —C(═O)O— and X and Y are H, the repeating units having a carboxyl group or a blocked carboxyl group, and coupling the carboxyl group or blocked carboxyl group with a fluorocarbon or a material that binds specifically to a biomolecule. The coupling reaction may be performed by a reaction with a functional group such as an amino group of molecules or an amino group introduced by functionalization. For example, the polymer including the repeating units of Formulae M1 and M2 having a carboxyl group or a blocked carboxyl group may be poly(acrylic acid) (PAA) or poly(methacrylic acid) (PMAA). For example, the solid support may be prepared by immobilizing the polymer including the repeating units of Formulae M1 and M2 having a carboxyl group or a blocked carboxyl group onto a solid support and sequentially or simultaneously coupling a fluorocarbon and a material specifically binding to a biomolecule. The immobilizing of the polymer onto a solid support may be performed using a known method of immobilizing a polymer onto a solid support having a reactive group. For example, the immobilizing process may be performed by reacting the polymer having a carbonyl group activated with carbodiimide with a solid support having a reactive amino group on its surface to immobilize the polymer on the solid support through an amide bond by the reaction between the amino group and the activated carbonyl group.

The coupling may be carried out as follows. For example, the coupling may be carried out by activating a reactive carboxyl group of the repeating units of Formulae M1 and M2 in a polymer, for example, an acrylate polymer such as poly(acrylic acid) (PAA), poly(methacrylic acid) (PMA), poly(methylacrylic acid) (PMAA), or poly(ethylacrylic acid) with carbodiimide (for example, ethyl-dimethylaminopropyl carbodiimide (EDC)), and then coupling the activated carbodiimide to an amino group on a surface of a solid support.

About 10% to about 90% of the repeating units of Formulae M1 and M2 in the polymer may includes a material specifically binding to the biomolecule. About 10% to about 90% of the repeating units of Formulae M1 and M2 in the polymer may include a fluorocarbon. In some embodiments, when at least one polymer may be immobilized on the solid support, at least one material specifically binding to a biomolecule may be immobilized on the solid support at a high density.

The polymer may include at least one of the repeating units represented by Formula M1 or M2, where W¹ and W² may be —C(═O)O—, X and Y may be H, and the polymer may be linked to the solid support via a carboxyl group of the repeating units of Formula M1 or M2.

In some embodiments, the polymer of the solid support may specifically bind to a biomolecule without non-specific binding of other biomolecules to form a biomolecule-solid support composite.

According to another aspect of the present disclosure, there is provided a polymer presently unbound to a solid support. The polymer may be the same as the polymer described above in the embodiments of the solid support.

According to another aspect of the present disclosure, a method of binding a biomolecule from a sample to a solid support, the method including contacting a solid support with the biomolecule from a sample to form a biomolecule-solid support composite; the solid support including at least one polymer immobilized thereon, the at least one polymer including at least one of repeating units represented by Formula M1 and at least one of repeating units represented by Formula M2:

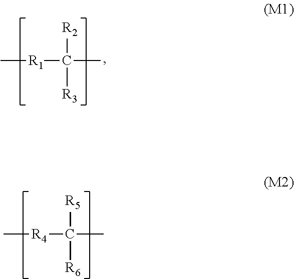

wherein, in Formulae M1 and M2,

R₁ and R₄ are each independently a bond or a substituted or unsubstituted C₁₋₂₀ alkyl, C₂₋₂₀ alkenyl, or C₂₋₂₀ alkynyl group;

R₂ and R₅ are each independently a hydrogen (H), a halo, or a substituted or unsubstituted C₁₋₂₀ alkyl, C₂₋₂₀ alkenyl, or C₂₋₂₀ alkynyl group;

R₃ is W¹—X;

R₆ is W²—Y;

W¹ and W² are each independently deleted, —C(═O)—, —C(═S)—, —C(═O)O—, —C(═O)O—C(═O)—, —C(═O)NR⁷—, —C(═S)NR⁷—, —S(═O)—, or —S(═O)₂—;

R⁷ is H or a C₁₋₂₀ alkyl group; and

X and Y are each independently selected from the group consisting of H, a fluorocarbon, and a material specifically binding to a biomolecule.

According to the method of binding a biomolecule of a sample to a solid support, non-specific binding of the material to the biomolecule of the sample may be reduced.

In some embodiments, the contacting may be carried out under conditions where the material of the polymer that specifically binds to a biomolecule may be bound to the biomolecule. For example, the contacting may be carried out in a liquid medium having pH, a salt concentration, and a temperature that are suitable for binding between the material and the biomolecule. The liquid medium may be water or a buffer (e.g. a PBS buffer). The pH may be a physiological pH, for example, in the range of about 6.8 to about 7.0. For example, the temperature may be in the range of about 15° C. to about 40° C. for example, in the range of about 15° C. to about 37° C. These reaction conditions may be appropriately selected by one of ordinary skill in the art according to selected biomolecule and material specifically binding thereto.

The polymer and the biomolecule referred to in the contacting may be the same as those described above in conjunction with the solid support according to an aspect of the present disclosure. The sample may be a certain biomolecule-containing sample. The sample may be obtained from a living body or contain artificially synthesized biomolecules. The biomolecule-polymer composite may be formed by binding between an antibody and an antigen, between a ligand and a receptor, or between an enzyme and a substrate, an inhibitor or activator of an enzyme, or a coenzyme.

The method may further include washing the biomolecule-polymer composite after the contacting. The washing may be performed to remove materials non-specifically bound to the biomolecule and/or the polymer, while the binding of the biomolecule-polymer composite is maintained. For example, the washing may be performed by flowing a liquid medium capable of removing the materials non-specifically bound to the biomolecule and/or the polymer, for example, water or a buffer (e.g., a PBS buffer), over the biomolecule-polymer composite, while the binding of the biomolecule-polymer composite is maintained.

The method may further include eluting the biomolecule from the biomolecule-polymer composite after the contacting. The eluting may be performed by flowing a liquid medium capable of removing the binding of the biomolecule-polymer composite over the biomolecule-polymer composite. The eluting may be performed using a liquid medium having given gradients of pH and/or salt concentration. An eluent used in the eluting may be a liquid medium that is appropriately given with gradients of pH and/or salt concentration according to a selected biomolecule and material specifically binding thereto, for example, water or a buffer (e.g., a PBS buffer). These eluting conditions may be appropriately selected by one of ordinary skill in the art according to the selected biomolecule and material specifically binding thereto. For example, if a protein is selected as the biomolecule and the material specifically binding thereto is an antibody, the eluting may be performed using a well-known method in the art such as a method of separating an antigen, for example, protein by affinity chromatography using an antibody, and the eluting conditions are obvious to one of ordinary skill in the art.

By using the method described above, specific biomolecules may be specifically bound to the polymer without binding of non-specific biomolecules, for example, protein thereto. A material containing a specific biomolecule, for example, a cell may be specifically bound to the polymer by specifically binding the specific biomolecule to the polymer.

By the method described above, specific biomolecules may be separated from a sample without binding of non-specific biomolecules, for example, protein thereto. A material containing a specific biomolecule, for example, a cell may be specifically separated from a sample by specifically binding the specific biomolecule to the polymer or separating the specific biomolecule therefrom.

In some embodiments, the method may further include: confirming whether the biomolecule of the sample is bound to the polymer; and determining that the biomolecule is in the sample when the biomolecule is confirmed as being bound to the polymer, or determining that the biomolecule is not in the sample when the biomolecule is confirmed as not being bound to the polymer.

The method may include confirming whether the biomolecules in a sample are bound to the polymer. The confirming process may be performed by detecting whether or not the biomolecule-polymer composite exists. The confirming process may be performed by eluting the biomolecules from the biomolecule-polymer composite. The detecting process may be performed using any of a variety of well-known methods in the art such as a spectroscopic method, an electric method, or enzyme-linked immunosorbent assay (ELISA).

In some embodiments, specific biomolecules may be specifically bound to the polymer immobilized on the solid support, without binding of non-specific biomolecules thereto.

In some embodiments, specific biomolecules may be specifically bound to the polymer or may be separated from a sample, without binding of non-specific biomolecules, for example, protein thereto. A material containing specific biomolecules may be specifically bound to the polymer or may be separated therefrom, by specifically binding the specific biomolecule to the polymer or separating the specific biomolecule therefrom. A material including specific biomolecules may be enriched.

In some other embodiments, whether biomolecules are in a sample may be efficiently detected.

One or more embodiments of the present disclosure will now be described in detail with reference to the following examples. However, these examples are only for illustrative purposes and are not intended to limit the scope of the one or more embodiments of the present disclosure.

### Example 1

### Manufacture of Solid Support Including Polymer

To manufacture a solid support including a polymer, according to an embodiment of the present disclosure, poly(acrylic acid) (PAA) including the repeating units of Formulae M1 and M2, where R₁ and R₄ are —CH₂—, R₂ and R₅ are H, R₃ and R₆ are —C(═O)OH, was immobilized on a surface of a solid support. Beads were used as the solid support.

Subsequently, 80 μg of Protein G (available from Sigma-Aldrich) per 100 μl of the solid support was introduced and bound to PAA, followed by adding fluorocarbon and binding the same to PAA. To this end, a carboxyl group of PAA was previously activated with carbodiimide. Detailed methods of preparing solid supports including polymer, according to embodiments of the present disclosure, are as follows.

1.1 Selection of polymer

To reduce the amount of functional groups per unit area and steric hindrance, a polymer was introduced into a solid support. To select appropriate polymers that are hydrophilic and have low non-specific binding, relative contact angle measurements and bicin choninic acid (BCA) assay using a BCA protein assay kit (available from Pierce) to quantize the amount of protein adsorbed onto each protein were performed after coating gamma aminopropyltriethoxylsilane (GAPS) slides with PAA, poly(methacrylate) (PMA), poly(methylsilane) (PMS), and poly(ethyl methacrylate) (PEM), respectively.

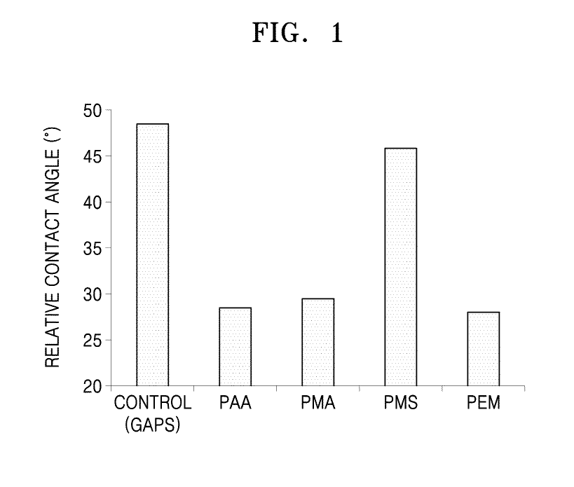

FIG. 1 is a graph of relative contact angles of PAA, PMA, PMS, and PEM. Referring to FIG. 1, the relative contact angles of PAA, PMA, and PEM were found to be smaller than the relative contact angle of a GAPS slide used as a control group.

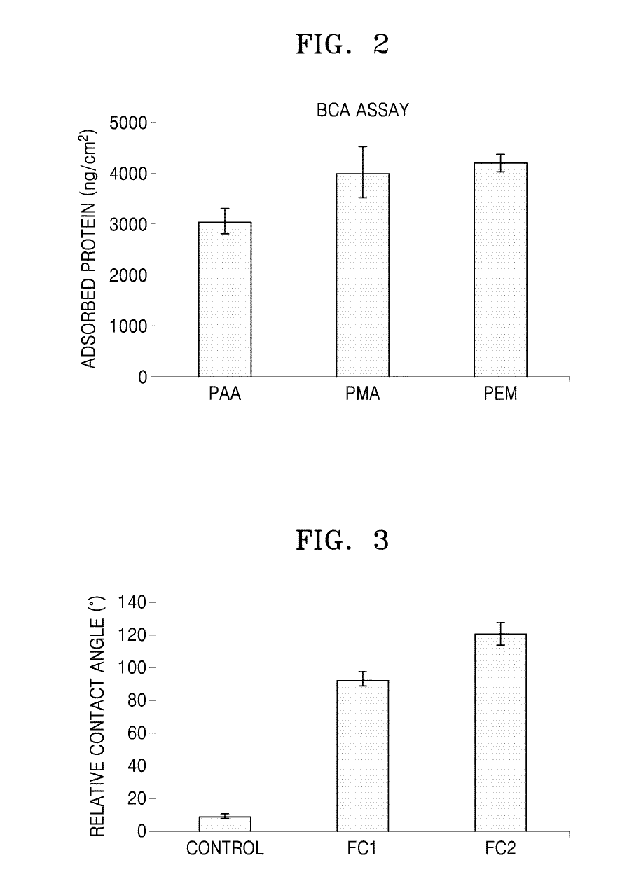

BCA assay was performed on the PAA, PMA and PEM having small relative contact angles. FIG. 2 is a graph illustrating the results of BCA assay on PAA, PMA, and PEM. Referring to FIG. 2, the amount of adsorbed protein on the PAA was found to be smaller than the adsorbed protein amounts of PMA and PEM.

1.2. Binding of Polymer Having Carboxyl Group to Magnetic Bead

Magnetic beads were used as the solid support. Dynabeads® M-270 Amine (available from Invitrogen) were used. Dynabeads® M-270 Amine are uniform, superparamagnetic beads composed of highly cross-linked polystyrene with magnetic material precipitated in pores evenly distributed throughout the beads. The beads are further coated with a hydrophilic layer of glycidyl ether to seal iron oxide inside the beads, with the surfaces thereof activated with primary amino functional groups on a short hydrophilic linker.

The hydrophilic surface ensures low non-specific binding, good dispersion abilities, and easy handling of the beads in a wide variety of buffers. The beads are commercially available as an aqueous suspension at a concentration of 2×10⁹ beads/ml (approximately 30 mg/ml). The diameter of the beads was 2.8 μm. The surface-reactive primary amino groups allow immobilization of ligands such as proteins, peptides, carbohydrates or other target specific molecules.

100 μl of Dynabeads® M-270 Amine (available from Invitrogen) were washed twice with 200 μl of 0.1M 2-(N-morpholino)ethanesulfonic acid (MES), 0.5 M NaCl, and a pH 6 buffer and then resuspended in 100 μl of the same buffer. Poly(acrylic acid) (PAA) selected in Section (1) was added as a polymer. 48 μl of a 1:10 diluted solution of a PAA solution (35% w/v in water, average weight molecular weight (M_{w}) of about 15,000 Da, catalog No.: 416037, available from Aldrich) and 236 μl of the buffer were mixed together, and the resultant mixture was added to the resuspended bead solution and mixed well.

Next, 54 μl of a solution of 75 mg/ml of 1-ethyl-3-(3-dimethylaminopropyl)carbodiimide (EDC) dissolved in distilled water and 210 μl of a solution of 15 mg/ml of N-hydroxysuccinimide (NHS) dissolved in distilled water were added to the mixture, and the resulting mixture was rotated for 1 about hour. Subsequently, the mixture was washed twice with 400 μl of 0.5M NaCl buffer (pH 6.0) and resuspended in 400 μl of the same buffer. As a result, magnetic beads (hereinafter, also referred to as “AP”) with PAA bound to the surfaces by an amide bond were obtained. The amide bond was formed by binding between a carbonyl group of PAA and primary amino groups of the magnetic beads.

1.3. Binding of Protein G to Magnetic Bead Surfaces with PAA Bound Thereon

The bead suspension of Example 1 (prepared according to Section 1.2) was washed twice with 400 μl of 0.025M MES buffer (pH 6.0). Subsequently, 236 μl of the same buffer, 54 μl of an EDC solution (75 mg/ml EDC in 0.025M MES buffer, pH 6.0), and 210 μl of an NHS solution (15 mg/ml of NHS in 0.025M MES buffer, pH 6.0) were added to the resultant bead suspension and mixed together, and the resultant mixture was then rotated for 30 minutes.

The magnetic beads were washed twice with 400 μl of the same buffer and resuspended in 400 μl of the same buffer. Then, 3 μl of a protein G solution (10 μg/μl) (P4689, available from Sigma-aldrich) was added to the bead suspension and the resultant mixture was rotated for 1 hour.

As a result, magnetic beads (hereinafter, also referred to as “APpG”) with Protein G-bound PAA on the surfaces thereof were obtained. Protein G may was bound to the magnetic beads via a carbonyl group of PAA.

1.4 Binding of Blocker to Magnetic Bead Surfaces with Protein G-Bound Poly(Acrylic Acid) Thereon

Fluorocarbon as a blocker was added to the magnetic beads prepared according to Section 1.3 of Example 3, and bound thereto. A linear fluorocarbon represented by CF₃(CF₂)₇CH₂NH₂ (hereinafter, also referred to as ‘FC1’) and a branched fluorocarbon represented by CF₃CF₂CF₂CH₂NH₂ (hereinafter, also referred to as ‘FC2’) were selected, and relative contact angles thereof were measured. Polydimethlysiloxane (PDMS) was coated on glass slides. Then, each of polyacrylic acid (PAA), FC1, and FC2 was applied on the PDMS of the glass slides. Contact angle of each of the above prepared glass slides with respect to water was measured by using a contact angle measurement apparatus (DSA30, KRUSS company) while each of the glass slides was in contact with water. FIG. 3 is a graph of relative contact angles of CF₃(CF₂)₇CH₂NH₂ (FC1) and CF₃CF₂CF₂CH₂NH₂ (FC2). In FIG. 3, the relative contact angle of control was measured to be less 5°. A more accurate measurement of the relative contact angle of the control was not available. Referring to FIG. 3, CF₃(CF₂)₇CH₂NH₂ (FC1) and CF₃CF₂CF₂CH₂NH₂ (FC2) were found to have high relative contact angles and be hydrophobic.

The blockers CF₃(CF₂)₇CH₂NH₂ (FC1) and CF₃CF₂CF₂CH₂NH₂ (FC2) were added to the magnetic beads of Example 1 (Section 1.3) with Protein G-bound PAA on the surfaces thereof, and coupled to the PAA backbone, thereby manufacturing magnetic beads with either one of the blockers and Protein G-bound PAA bound on the surfaces thereof. In particular, magnetic beads (hereinafter, also referred to as ‘APpGFC1’) with both CF₃(CF₂)₇CH₂NH₂ (FC1) and Protein G bound to the backbone of PAA on the surfaces thereof were manufactured by adding CF₃(CF₂)₇CH₂NH₂ (FC1) as a blocker to the magnetic beads of Example 1 with Protein G-bound PAA on the surfaces thereof and coupling the blocker thereto, and magnetic beads (hereinafter, also referred to as ‘APpGFC2’) with both CF₃CF₂CF₂CH₂NH₂ (FC2) and Protein G bound to the backbone of PAA on the surfaces thereof were manufactured by adding CF₃CF₂CF₂CH₂NH₂ (FC2) as a blocker to the magnetic beads of Example 1 with Protein G-bound PAA on the surfaces thereof and coupling the blocker thereto. The addition of fluorine (F) atom to the magnetic beads was characterized using time-of-flight secondary ion mass spectrometry (TOF-SIMS) for surface characterization. As a result, the relative amount of F was higher in the order of the magnetic beads with CF₃CF₂CF₂CH₂NH₂ (FC2), the magnetic beads with CF₃(CF₂)₇CH₂NH₂ (FC1), and the magnetic beads with FC1 or FC2 (control group). The amounts of C atom and oxygen atoms were the same in the three groups.

### Example 2

### Binding of Biomolecule and Solid Support Including Polymer

20 μl of the magnetic beads (‘APpGFC2’) with CF₃CF₂CF₂CH₂NH₂ (FC2) and Protein G bound to the backbone of PAA on the surfaces thereof were mixed with 200 ul of a buffer (w/BSA (5 g/100 ml)) and then incubated for about 2 hours. The magnetic beads (APpG) with Protein G-bound PAA on the surfaces thereof, and commercially available magnetic beads (Dynabeads® Protein G, available from Life Technologies) were used as control groups. After the incubating, the degree of non-specific adsorption on the polymer-including solid supports were evaluated by sodium dodecyl sulfate polyacrylamide gel electrophoresis (SDS-PAGE).

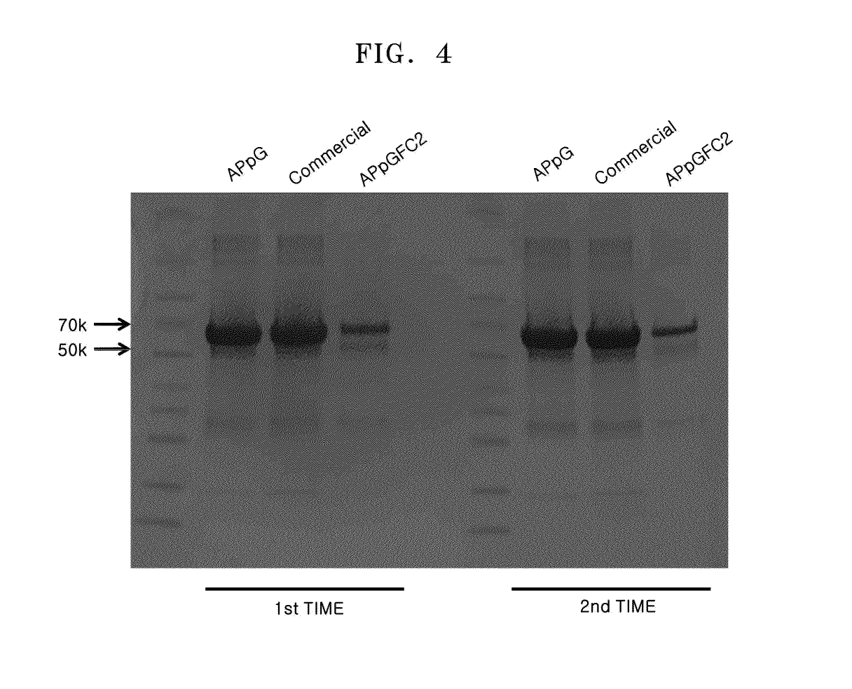

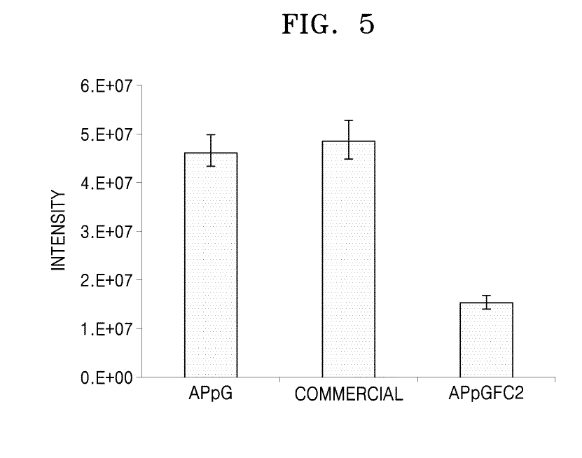

FIG. 4 is a graph illustrating the results of SDS-PAGE on the polymer-including solid supports APpGFC2 and APpG, and the commercially purchased solid support. FIG. 5 is a graph of band intensity resulting from SDS-PAGE on the tested three types of polymer-including solid supports after the incubation with BSA. Referring to FIGS. 4 and 5, the solid support APpGFC2 were found to exhibit a lower band intensity compared to the solid support ApPG and the commercial product. This is attributed to that the blocker FC2 included in the polymer (PAA) of the magnetic beads APpGFC2 blocked non-specific binding to the polymer due to the low surface energy, high hydrophobicity, and protein-repellent behavior of fluorocarbon.

### Example 3

### Binding of Biomolecule and Solid Support Including Polymer

3.1 Preparation of Magnetic Beads with Biotin-Coupled Protein G and PAA Bound to Surfaces Thereof

The bead suspension prepared according to Section 1.2 of Example 1 was washed twice with 400 μl of 0.025M MES buffer (pH 6.0). Subsequently, 236 μl of the same buffer, 54 μl of an EDC solution (75 mg/ml EDC in 0.025M MES, pH 6.0 buffer), and 210 μl of an NHS solution (15 mg/ml of NHS in 0.025M MES, pH 6.0 buffer) were added to the resultant bead suspension and mixed together, and the resultant mixture was then rotated for 30 minutes.

The magnetic beads were washed twice with 400 μl of the same buffer and resuspended in 400 μl of the same buffer. Then, 3 μl of a biotin (B)-coupled protein G solution (10 μg/μl) (available from Sigma-aldrich, P8045) was added to the bead suspension, and the resultant mixture was rotated for about 1 hour. As a result, magnetic beads (hereinafter, also referred to as ‘APpGB’) with biotin-coupled Protein G-bound PAA on the surfaces thereof were obtained.

Then, the blocker CF₃CF₂CF₂CH₂NH₂ (FC2) was added to the magnetic beads (APpGB) and coupled to the backbone of PAA bound to the surfaces of the magnetic beads (APpGB), thereby manufacturing APpGBFC2 with the block (FC2) and biotin-coupled Protein G bound on the backbone of PAA on the surfaces thereof.

3.2 Binding Between Polymer and Target Material

A sample including streptavidin as a target material was bound to the polymer of the magnetic beads (solid supports) to evaluate binding efficiencies between the polymer and streptavidin and degrees of non-specific binding.

20 μl of the magnetic beads (APpGBFC2) of Example 3 (prepared according to Section 3.2), 20 μl of the magnetic beads (APpGB) of Example 3 (prepared according to Section 3.1), and 20 μl of the magnetic beads (APpGFC2) of Example 1 (prepared according to Section 1.4) were each mixed with 200 ul of buffers (w/BSA (5 g/100 ml)) and 4 ug of streptavidin (available from Sigma-aldrich, S4762), and then incubated for about 2 hours, followed by SDS-PAGE.

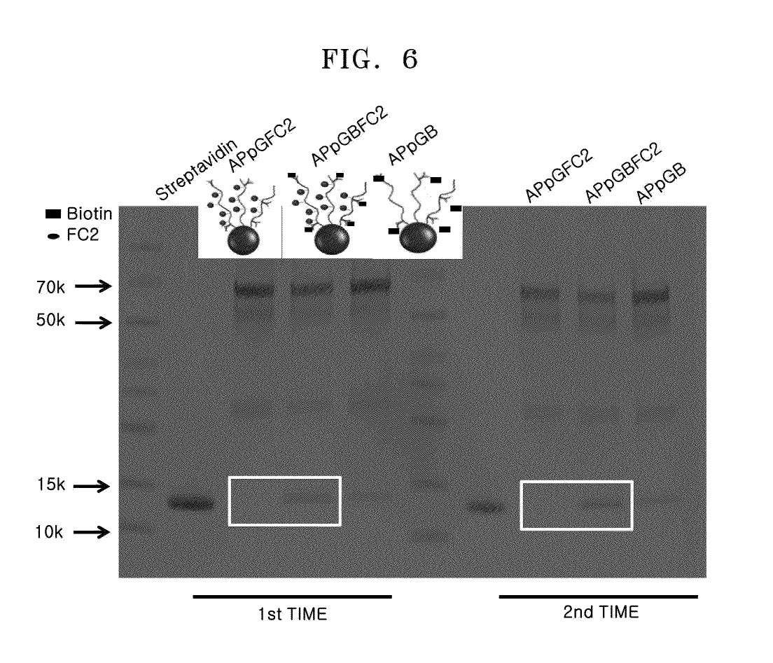

FIG. 6 illustrates the results of SDS-PAGE on the magnetic beads (APpGBFC2), the magnetic beads (APpGB), and the magnetic beads (APpGFC2) of Example 1 (prepared according to Section 1.4) after the incubation with the BSA-including buffer and streptavidin. In FIG. 6, specific bands indicating the specific binding of biotin and streptavidin are denoted by red boxes, and smaller circles around the magnetic beads (APpGFC2 and APpGBFC2) represented by larger circles denote fluorocarbon (FC2) used as a blocker. Binding efficiencies between streptavidin and the polymer-including solid supports (magnetic beads) were evaluated by SDS-PAGE after the incubation.

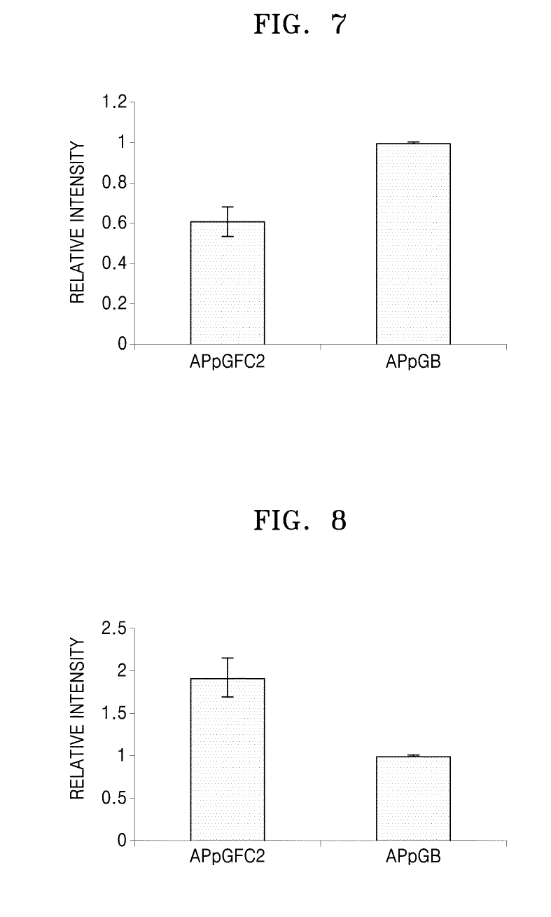

FIG. 7 is a graph of relative band intensity resulting from SDS-PAGE on the polymer-including solid supports (APpGBFC2 and APpGB) after the incubation with BSA. Referring to FIG. 7, the magnetic beads APpGBFC2 were found to exhibit a band intensity lower by about 40% than the magnetic beads ApPGB. This is attributed to that the blocker FC2 included in the polymer (PAA) of the magnetic beads APpGBFC2 blocked non-specific binding to the polymer due to the low surface energy, high hydrophobicity, and protein-repellent behavior of fluorocarbon.

Binding efficiencies between streptavidin as a specific protein and the polymer of the solid supports (APpGBFC2 and ApPGB) were evaluated by SDS-PAGE after the incubation. FIG. 8 is a graph of relative band intensities of streptavidin-solid support composites, resulting from SDS-PAGE on the solid supports (APpGBFC2 and APpGB) after the incubation with streptavidin. Referring to FIG. 8, the magnetic beads APpGBFC2 were found to exhibit a band intensity higher by about 190% than the magnetic beads ApPGB, indicating enhanced specific binding strength due to the reduced non-specific binding.

It should be understood that the exemplary embodiments described therein should be considered in a descriptive sense only and not for purposes of limitation. Descriptions of features or aspects within each embodiment should typically be considered as available for other similar features or aspects in other embodiments.

While one or more embodiments of the present invention have been described with reference to the figures, it will be understood by those of ordinary skill in the art that various changes in form and details may be made therein without departing from the spirit and scope of the present invention as defined by the following claims.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A solid support on which at least one polymer is immobilized, the at least one polymer comprising at least one of repeating units represented by Formula M1 and at least one of repeating units represented by Formula M2: 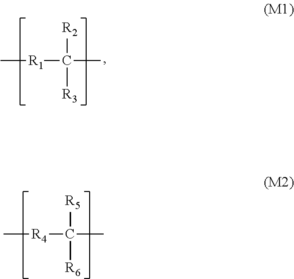
wherein, in Formulae M1 and M2,
R₁ and R₄ are each independently a bond or a substituted or unsubstituted C₁₋₂₀ alkyl, C₂₋₂₀ alkenyl, or C₂₋₂₀ alkynyl group;
R₂ and R₅ are each independently a hydrogen, a halogen, or a substituted or unsubstituted C₁₋₂₀ alkyl, C₂₋₂₀ alkenyl, or C₂₋₂₀— alkynyl group;
R₃ is W¹—X;
R₆ is W²—Y;
W¹ and W² are each independently a bond, —C(═O)—, —C(═S)—, —C(═O)O—, —C(═O)O—C(═O)—, —C(═O)NR⁷—, —C(═S)NR⁷—, —S(═O)—, or —S(═O)₂—,
R⁷ is H or a C₁₋₂₀ alkyl group;
X and Y are each independently selected from the group consisting of H, a fluorocarbon, and a material that specifically binds to one or more biomolecules.

2. The solid support of claim 1, wherein the fluorocarbon is a substituted or unsubstituted, linear or branched compound of fluoro-containing C₁₋₂₀.

3. The solid support of claim 1, wherein the fluorocarbon is a fluoro-containing C₁₋₂₀ alkyl compound, a fluoro-containing C₁₋₂₀ carbonyl compound, a fluoro-containing C₁₋₂₀ alkoxy compound, or a combination thereof.

4. The solid support of claim 1, wherein the biomolecule is selected from the group consisting of a protein, a nucleic acid, and a sugar.

5. The solid support of claim 1, wherein the material specifically binding to a biomolecule is selected from the group consisting of a protein, a nucleic acid, a sugar, and a cell.

6. The solid support of claim 1, wherein the material specifically binding to a biomolecule is an antibody, an antigen against an antibody, a receptor against a ligand, a ligand against a receptor, a substrate or inhibitor of an enzyme, or an enzyme against a substrate or inhibitor.

7. The solid support of claim 1, wherein the material specifically binding to a biomolecule is Protein G, Protein A, lectin, an antibody, avidin, streptavidin, a receptor protein, or a combination thereof.

8. The solid support of claim 1, wherein the solid support comprises a bead, a plate, or a well on which the at least one polymer is immobilized.

9. The solid support of claim 1, wherein about 10% to about 90% of the number of repeating units of the polymer comprises at least one material that specifically binds to a biomolecule.

10. The solid support of claim 1, wherein about 10% to about 90% of the number of repeating units of the polymer comprises a fluorocarbon.

11. The solid support of claim 1, wherein the polymer comprises about 1 to about 300 repeating units of Formula M1 and about 1 to about 300 repeating units of Formula M2.

12. The solid support of claim 1, wherein, except for the repeating units of Formulae M1 and M2 directly bound to the solid support, R₁ and R₄ are —CH₂—, R₂ and R₅ are —H, W¹ and W² are —C(═O)NR⁷—, and X and Y are each independently selected from the group consisting of H, a fluorocarbon, and a material specifically binding to a biomolecule.

13. The solid support of claim 1, wherein
(a) the polymer comprises a repeating unit of Formula M1 where W¹ is —C(═O)O—, X is H to provide a carboxyl group;
(b) the polymer comprises a repeating unit of Formula M2 where W² is —C(═O)O—, Y is H to provide a carboxyl group;
or both (a) and (b);
and the solid support is bonded to the polymer via the carboxyl group of the repeating units of Formula M1 or M2.

14. A method of binding a biomolecule to a solid support, the method comprising contacting a solid support according to claim 1 with a biomolecule to form a biomolecule-solid support composite, wherein the polymer of the solid support of claim 1 includes at least one repeating subunit of M1 or M2 in which X or Y or both X and Y comprise a material that specifically binds to the biomolecule.

15. The method of claim 14, wherein non-specific binding of the materials to the solid support is reduced compared to a polymer.

16. The method of claim 14, further comprising washing the biomolecule-solid support composite after contacting the solid support with the biomolecule.

17. A method of detecting a biomolecule in a sample comprising binding a biomolecule from a sample to a solid support according to claim 14 to form a biomolecule-solid support composite, and eluting the biomolecule from the biomolecule-solid support composite.

18. The method of claim 17, further comprising washing the biomolecule-solid support composite before eluting the biomolecule from the biomolecule-solid support composite.

19. The method of claim 17, further comprising:
determining that the biomolecule is present in the sample when the biomolecule is identified as being bound to the polymer, or determining that the biomolecule is not in the sample when the biomolecule is identified as not being bound to the polymer.
